(19) United States
(12) Patent Application Publication
(10) Pub. No.: US 2003/0215834 A1
(43) Pub. Date: Nov. 20, 2003

(54) Method of ordering synthesis of primer used for gene amplification, program therefor and recording medium of the program

(75) Inventors: Masaru Kaneko, Tokyo (JP); Yoshiaki Ikeda, Kawasaki (JP); Seiichi Hatagami, Kawasaki (JP)

Correspondence Address: STAAS & HALSEY LLP, SUITE 700, 1201 NEW YORK AVENUE, N.W., WASHINGTON, DC, 20005, US

(73) Assignee: Fujitsu Limited, Kawasaki (JP)

(21) Appl. No.: 10/331,501

(22) Filed: Dec. 31, 2002

(30) Foreign Application Priority Data
May 15, 2002 (JP) 2002-139668

(51) Int. Cl. C12Q 1/68; G06F 19/00; G01N 33/48; G01N 33/50

(52) U.S. Cl. 435/6; 702/20

(57) ABSTRACT

A method of ordering synthesis of a primer used for gene amplification includes default displaying parameter information of a primer used for amplification based on the input information of a target gene, displaying the results of setting a primer for the amplification of the target gene based on the selected parameter information of a primer, default displaying primer designing information of the basis of the selected results of setting a primer, default displaying information regarding the type, length and purity of a primer used for amplification from the selected primer designing information, and ordering the synthesis of a primer based on the selected information regarding the type, length and purity of a primer.

### BACKGROUND OF THE INVENTION

1. Field of the Invention

This invention relates to a method of ordering synthesis of a primer used for gene amplification, a program therefor and a recording medium of the program, a method of ordering a reagent kit used for detection of gene amplification level, a program therefor and a recording medium of the program, and an apparatus for ordering synthesis of a primer and a reagent kit, a program therefor and a recording medium of the program. Especially, this invention relates to a method of ordering synthesis of a designed primer, a method of ordering a reagent kit used for detecting gene amplification level using a primer, an apparatus for ordering synthesis of a primer and a regent kit, a program for realizing these methods or the apparatus, and a recording medium for recording the program.

2. Description of the Related Art

In recent years, the gene amplification technique essential to experiments associated with a gene or gene diagnosis has been progressing. Examples of techniques for gene amplification include the PCR method, and other amplification methods such as the LAMP method (Eiken Chemical Co., Ltd.) (International Publication No. WO00/28082). For example, where DNA is amplified by the LAMP method, reagents comprising 4 types of primers (FIP Primer, F3 Primer, BIP Primer, B3 Primer), strand displacement DNA polymerase, a substrate (deoxynucleotide triphosphate) and a reaction buffer are needed. An amplification product or the presence or absence of amplification can be detected by incubating a target gene with the above described reagents for 1 hour.

Previously, genome researchers have produced primer designing software by themselves, and based on the designing results, they have ordered the synthesis of a primer or have ordered a reagent kit used for the detection of gene amplification level to traders interested, which the researchers have known. However, since the type, length and purity of a primer, which are parameter information for designing a primer, and a reagent kit used for the detection of gene amplification level, have astronomical numbers of combination patterns, primer designers had to depend on trial-and-error methods to find the optimal parameter from among these astronomical numbers of combination patterns to order primer synthesis and a reagent kit. Thus, researchers themselves have sought the optimal input parameter for every research, and accordingly there have been problems such that an ordering mistake is made, the results of research vary, and that the cause of the failure of research cannot be found.

### SUMMARY OF THE INVENTION

It is an object of the present invention to provide a method of ordering synthesis of a primer used for gene amplification, a program therefor and a computer readable recording medium of the program, which overcome the above problems of the prior art technique, and with which, on the basis of the results of designing a primer, a genome researcher selects parameter information while referring to a default display (a content displayed on a screen as a default or an initial value), and then the primer designing information is seamlessly connected with the order of the synthesis of a primer used for gene amplification and the order of a reagent kit.

Moreover, it is another object of the present invention to provide a method of ordering a reagent kit used for detection of gene amplification level, a program therefor and a computer readable recording medium of the program, with which, on the basis of the results of designing a primer, a genome researcher selects parameter information while referring to a default display, and then the primer designing information is seamlessly connected with the order of the synthesis of a primer used for gene amplification and the order of a reagent kit.

Furthermore, it is another object of the present invention to provide an apparatus for ordering synthesis of a primer and a reagent kit, a program therefor, and a computer readable recording medium of the program, with which, on the basis of the results of designing a primer, a genome researcher selects parameter information while referring to a default display, and then the primer designing information is seamlessly connected with the order of the synthesis of a primer used for gene amplification and the order of a reagent kit.

To achieve the above described objects, the method of ordering synthesis of a primer used for gene amplification of the present invention comprises the steps of receiving an attribute information of a primer, extracting a reagent information corresponding to the received attribute information from a plurality of pair of an attribute information and an reagent information corresponding to each other based on the received attribute information, and sending the extracted reagent information.

The method of ordering synthesis of a primer used for gene amplification of the present invention comprises the steps of default displaying (displaying as a default or an initial value) parameter information of a primer used for amplification based on input information of a target gene, displaying the results of setting a primer used for amplification of the target gene based on selected parameter information of a primer, default displaying primer designing information based on the selected results of setting a primer, default displaying information regarding the type, length and purity of a primer used for amplification from the selected primer designing information, and ordering the synthesis of a primer based on the selected information regarding the type, length and purity of a primer.

Preferably, in the above described method of ordering synthesis of a primer used for gene amplification, the above described step of default displaying the above primer designing information further comprises default displaying the information by referring order historical data in which primer synthesis ordering information is recorded.

Moreover, the program of the present invention is a program to cause a computer to perform a processing of ordering synthesis of a primer used for gene amplification, and it causes a computer to perform each of the above described processes.

Furthermore, the computer readable recording medium of the present invention is a computer readable recording medium recording a program to cause a computer to perform a processing of ordering synthesis of a primer used for gene amplification, and it records the program to cause a computer to perform each of the above processes.

The method of ordering a reagent kit for detecting gene amplification level using a primer of the present invention comprises the steps of receiving information regarding the type, length and purity of a primer ordered to be synthesized, searching through a database table which is previously prepared based on the received information, default displaying reagent kit information to detect gene amplification level based on the results of the searching, and ordering a reagent kit based on reagent kit information which is selected from the default displayed contents or input separately.

Preferably, in the above described method of ordering a reagent kit used for detection of gene amplification level, the above described step of default displaying reagent kit information used for the detection of gene amplification level further comprises default displaying information of a first reagent kit and information of a second reagent kit, the information of a first reagent kit containing a primer having a high amplification level and containing amplification level confirmation information calculated based on the type, length and purity of a primer, the information of a second reagent kit containing a primer having a high amplification level in a case that a different purity is applied,

Preferably, in the above described method of ordering a reagent kit used for detection of gene amplification level, corresponding information between information regarding the type, length and purity of a primer and reagent kit information is recorded on the database table.

Moreover, the program of the present invention is a program to cause a computer to perform a processing of ordering a reagent kit to detect gene amplification level using a primer, and it causes a computer to perform each of the above described processes.

Furthermore, the computer readable recording medium of the present invention is a computer readable recording medium recording a program to cause a computer to perform a processing of ordering a reagent kit to detect gene amplification level using a primer, and it records the program to cause a computer to perform each of the above described processes.

The apparatus for ordering the synthesis of a primer and for ordering a reagent kit of the present invention comprises input information receiving means for receiving parameter information necessary for the order of the synthesis of a primer and the order of a reagent kit, both of the orders being input by a user, database searching means for searching through a database table which is previously prepared based on the received input information, output information displaying means for default displaying output information obtained as a result of the searching process to the user's terminal, and ordering information sending means for sending primer synthesis ordering information and reagent kit ordering information via a network, both of the information being received from the user.

Preferably, in the above described apparatus for ordering synthesis of a primer and for ordering a reagent kit, corresponding information between information regarding the type, length and purity of a primer and reagent kit information is recorded on the database table.

Moreover, the program of the present invention is a program to cause a computer to perform a primer synthesis ordering processing and a reagent kit ordering processing. The program causes the computer to perform receiving parameter information necessary for order of synthesis of a primer and order of a reagent kit, both of the orders being input by a user, searching through a database table which is previously prepared based on the received input information, default displaying output information obtained as a result of the searching process to the user's terminal, and sending primer synthesis ordering information and reagent kit ordering information via a network, both of the information being received from the user.

Furthermore, the computer readable recording medium of the present invention is a computer readable recording medium recording a program to cause a computer to perform a primer synthesis ordering processing and a reagent kit ordering processing, and it records the program to cause a computer to perform each of the above described processes.

According to the present invention, genome researchers can set a parameter on a portal by a selecting and clicking method according to a default display, and accordingly they can order the synthesis of a primer and a reagent kit, which are necessary and optimal for their research. As a result, it becomes possible to reduce variation of research results and it also becomes possible to pursue the cause of the failure of research.

### BRIEF DESCRIPTION OF THE DRAWINGS

FIG. 1 is a schematic diagram of the method of ordering a primer used for gene amplification of the present invention.

FIG. 2 is a view showing an example of the system configuration figure of an ordering apparatus 2.

FIG. 3 is a view showing the flow of a primer synthesis ordering processing.

FIG. 4 is a view showing an example of a parameter setting screen.

FIG. 5 is a view showing an example of a designing information confirmation screen.

FIG. 6 is a view showing an example of a designing information confirmation screen.

FIG. 7 is a view showing an example of a synthesis ordering screen.

FIG. 8 is a view showing the flow of a price determination processing.

FIG. 9 is a view showing the flow of a reagent kit ordering processing.

FIG. 10 is a view showing an example of a reagent kit ordering screen.

FIG. 11 is a view showing an example of a reagent kit determination table.

### DETAILED DESCRIPTION OF THE PREFERRED EMBODIMENTS

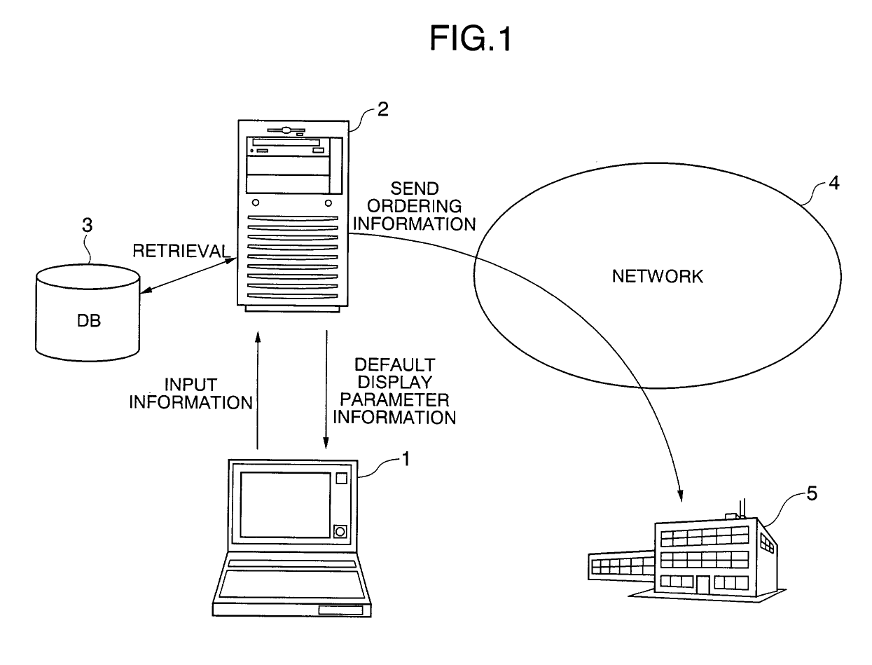

FIG. 1 is a view showing an example of the method of ordering a primer used for gene amplification of the present invention. 1 denotes a user's terminal, and 2 denotes an ordering apparatus for receiving information input from the terminal 1, searching through a database (DB) 3, on-screen displaying output information to the terminal 1, as well as ordering synthesis of a primer and ordering a reagent kit. 3 denotes a database (DB) which stores information of primer designing, information of a reagent kit, information of SNPs, etc. 4 denotes a network, and 5 denotes traders interested which receive an order from a user and perform the synthesis of a primer and the synthesis of a reagent kit.

In the present invention, the ordering apparatus 2, which received information input from the terminal 1, searches though the table of the database (DB) 3, and default displays (displays as a default or an initial value) to the terminal 1, input parameter information necessary for the rational order of the synthesis of a primer and a reagent kit. Then, based on the parameter selected by the user at the terminal 1, information of the order of primer synthesis and information of the order of a reagent kit are sent to the traders interested 5 via the network 4.

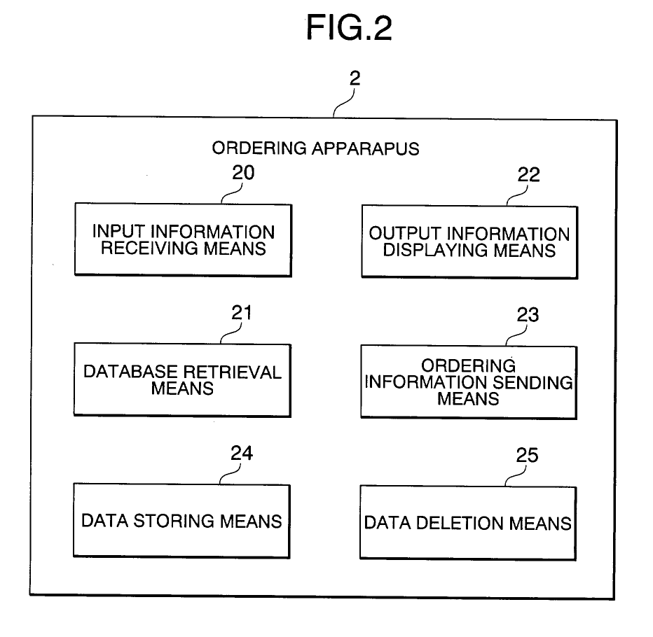

FIG. 2 is an example of the system configuration figure of the ordering apparatus 2. 20 denotes input information receiving means for receiving information input from the terminal 1, 21 denotes database searching means for searching through the database (DB) 3, 22 denotes output information displaying means for default displaying to the terminal 1, information output as a result of the database searching, and 23 denotes ordering information sending means for sending primer synthesis ordering information or reagent kit ordering information received from the terminal 1, to the traders interested 5 via the network 4. 24 denotes data storing means for storing various data, and 25 denotes data deletion means for deleting various data.

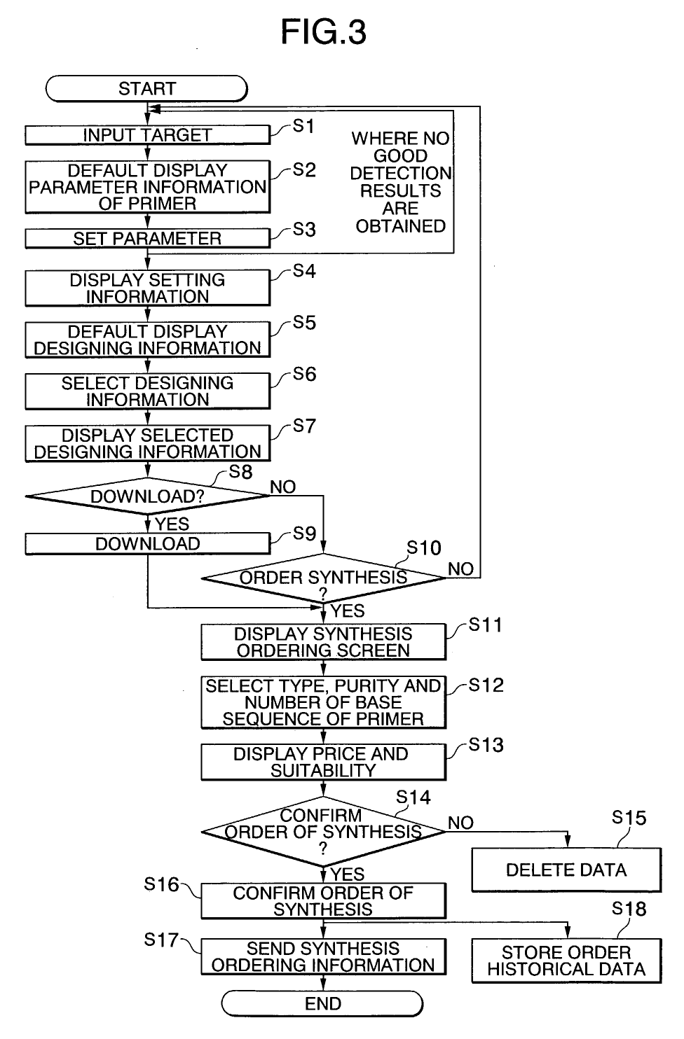

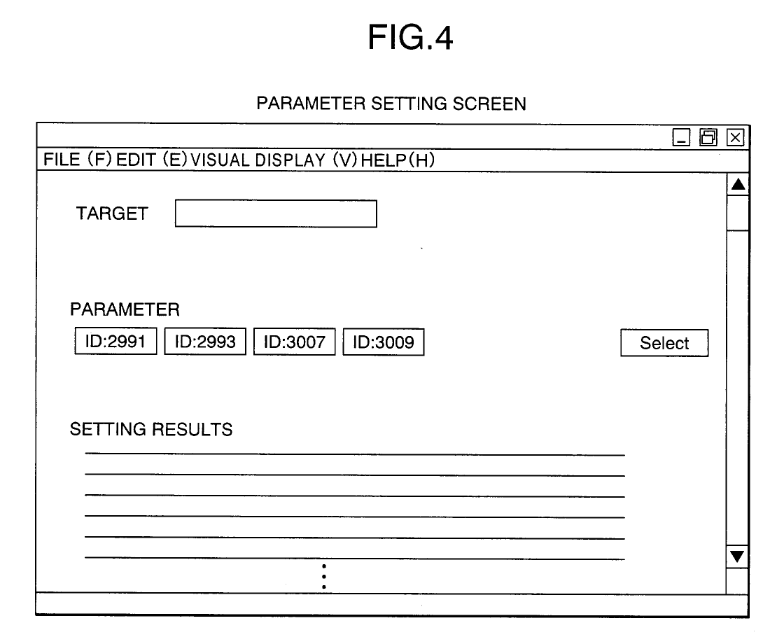

Based on FIG. 3, an example of the method of ordering the synthesis of a primer of the present invention will be explained below, while referring to FIGS. 4 to 8. FIG. 3 is a view showing the flow of a primer synthesis ordering processing of the present invention. When a user inputs information (target) on a target gene to be amplified on a parameter setting screen in FIG. 4 at the terminal 1 (step S1), the input information receiving means 20 of the ordering apparatus 2 receives the input target information, and then the database searching means 21 searches through the table of the DB 3. Then, the parameter information of a primer to be used for the amplification of the target gene, which is output as a result of the searching, is default displayed to the terminal 1 by the output information displaying means 22 (step S2). For example, on the parameter setting screen in FIG. 4, four parameters having primer ID numbers of 2991, 2993, 3007 and 3009, respectively, are default displayed.

Subsequently, parameter is selected and set at the user's terminal 1 (step S3). The parameter is set by clicking a “Select” button on the parameter setting screen in FIG. 4. The user can either select a parameter from parameters which are default displayed by clicking the “Select” button, or input a parameter on the user's own. When a parameter is set, parameter setting information is received by the input information receiving means 20 of the ordering apparatus 2 as with the above description, and after the database searching means 21 searches through the DB 3, the setting results are displayed on the parameter setting screen in FIG. 4 by the output information displaying means 22 (step S4). These setting results are considered as a specification (an alignment of base sequences) used for amplifying the target gene. Where a parameter providing no good detection results is set, setting results are not output, and the parameter setting screen is recalled (step S3).

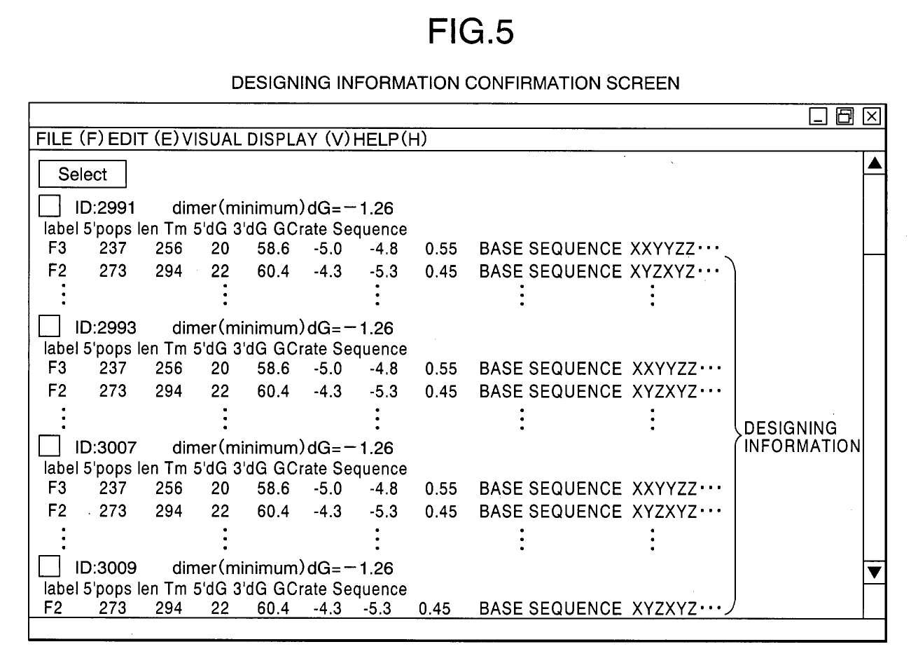

When the user clicks the “Select” button, a designing information confirmation screen in FIG. 5 is displayed to the terminal 1 by means of the ordering apparatus 2. On the designing information confirmation screen, primer ID defined as design No. and designing information (base sequences) on the basis of the above described parameter setting result are default displayed (step S5). The designing information has a configuration in which setting results as a specification are divided into the alignment of base sequences. In FIG. 5, 4 types of designing information corresponding respectively to primer ID Nos. 2991, 2993, 3007 and 3009 are default displayed, and the user can select and click designing information that the user intends to use.

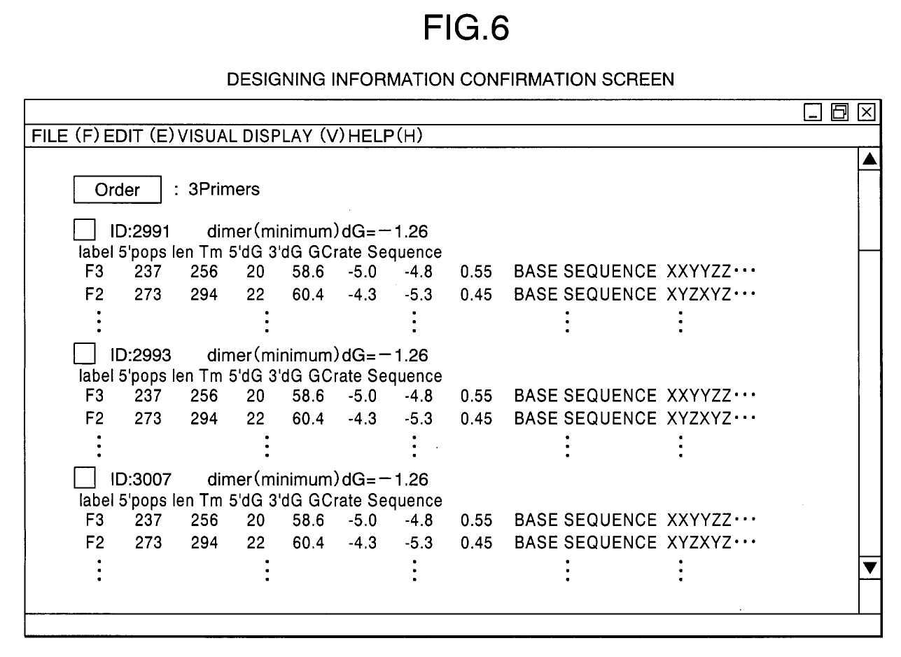

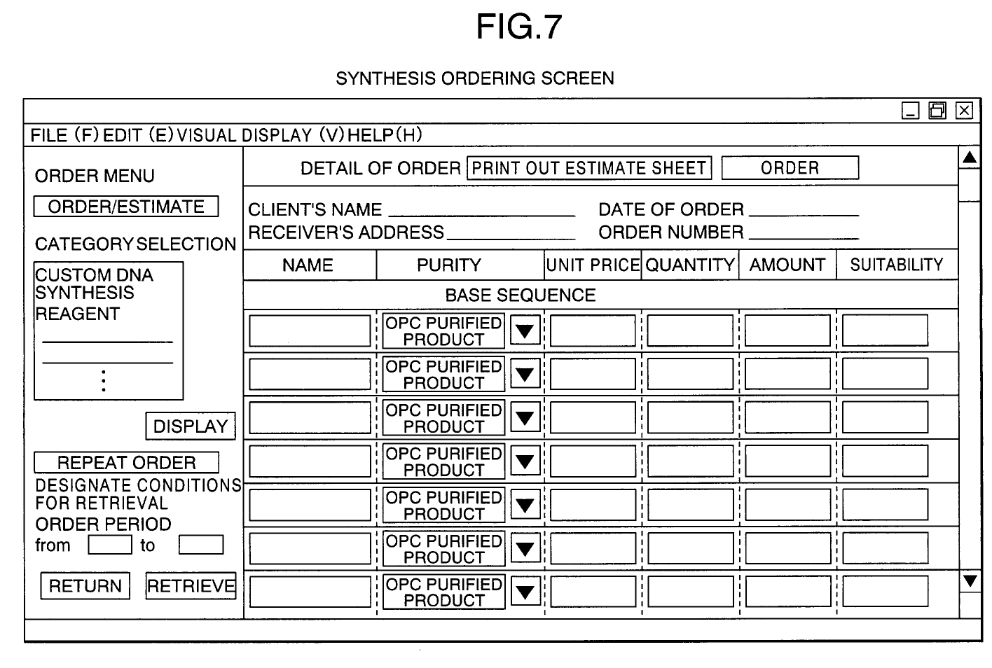

When the user selects designing information and clicks a “Select” button (step S6), as shown in FIG. 6, the selected designing information is displayed on the screen (step S7). In FIG. 6, the 3 types of designing information selected are displayed. Then, on the designing information confirmation screen, the user considers whether or not the user downloads the selected designing information (step S8). Where the user decides not to download the designing information, the user considers whether or not the user orders primer synthesis (step S1). In this case, when the user orders the primer synthesis, the user displays an ordering screen by clicking an “Order” button on the designing information confirmation screen in FIG. 6 and then the user displays a synthesis ordering screen in FIG. 7 by selecting “Custom DNA Synthesis” in Category Selection (step S11). When the user does not order the primer synthesis, the primer parameter setting screen in FIG. 4 is recalled (step S1).

In contrast, where the user downloads the selected designing information, the user downloads it by clicking a download button (step S9). The downloaded information includes a design number, a parameter, and information of designing results. The user can obtain designing information in the user's personal computer by downloading the information. Accordingly, in a case where certain designing information is output by inputting information of a certain target gene and a primer parameter, it becomes possible for the user to refer to the downloaded information the next time, change the parameter or the like as appropriate, and obtain different designing information, repeatedly. When the user downloads information, the synthesis ordering screen in FIG. 7 is displayed by means of the ordering apparatus 2.

On the synthesis ordering screen in FIG. 7, the type (name), the purity and the quantity of the base sequence of a primer according to the selected designing information are default displayed (step S11). Regarding purity, purity having a high amplification level is displayed. When the user selects the type, purity and quantity of each base sequence (step S12), price (unit price and total amount) and suitability are displayed by means of the ordering apparatus 2 (step S13).

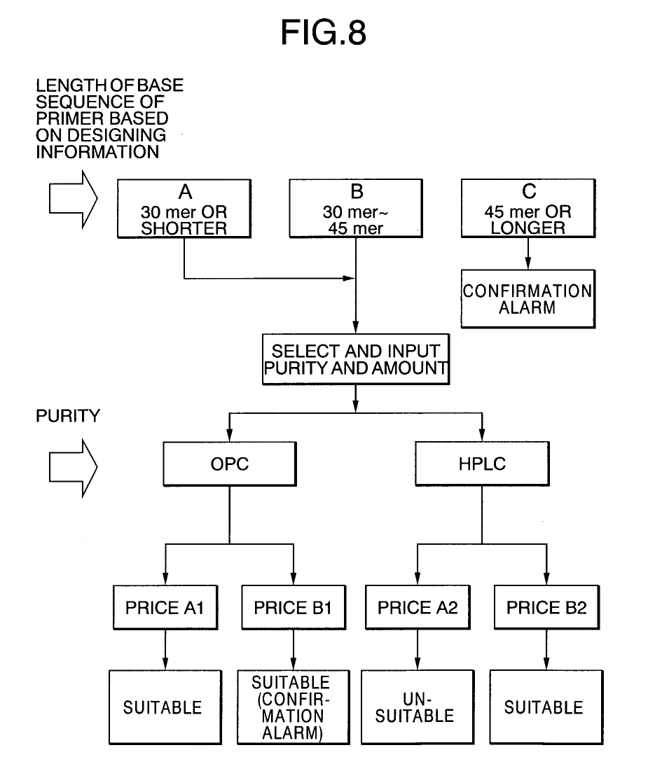

As shown in the flow of a price determination processing in FIG. 8, price is determined by the type, purity and quantity of the base sequence of a primer. For example, in FIG. 8, in the case of a base sequence A having a length of 30 mer or shorter, if OPC (low purity) is selected as purity, the price is determined as A1, but if HPLC (high purity) is selected as purity, the price is determined as A2. As another example, in the case of a base sequence B having a length of 30 mer to 45 mer, if OPC is selected as purity, the price is determined as BI, but if HPLC is selected as purity, the price is determined as B2. In the case of a base sequence C having a length of 45 mer or longer, an alarm indicating that the user should check the length of a base sequence is displayed. When price is determined as A1 or B2, “Suitable” is displayed. When price is determined as B1, it is suitable, but an alarm requiring the user to check the length of a base sequence is displayed. When price is determined as A2, “Unsuitable” is displayed.

Next, the user decides the confirmation of the order of primer synthesis (step S14), and then the user clicks an “Order” button on the synthesis ordering screen in FIG. 7 to confirm the order of the synthesis (step S16). By confirming the order of the synthesis, data including a design number, an order number, a base sequence ordered and an order name, are also confirmed. The ordering apparatus 2 receives the primer synthesis ordering information, which is input from the terminal 1. Then, by the ordering information sending means 23, the ordering apparatus 2 sends the information to the traders interested 5 via the network 4 (step S17).

In addition, when the order of primer synthesis is confirmed (step S16), order historical data including each of a design number, an order number, a quantity and an order name are stored by the data storing means 24 of the ordering apparatus 2 (step S18). This order historical data are deleted by the data deletion means 25, when delivery is confirmed. Moreover, where the confirmation of the order of primer synthesis is not carried out in the step of confirming the order of the synthesis (step S14), the data are deleted by the data deletion means 25 for security purpose (step S15).

In the present invention, since data downloaded by the user to the user's personal computer are linked with the order historical data by the design number, the user can confirm what contents of primer synthesis the user ordered, and for which design the order was made. Accordingly, even where the user intends to carry out the same experiment as the previous experiment, an order of primer synthesis having the same design contents can easily be made according to a default display (a content displayed as a default or an initial value), thereby avoiding the variation of experiment results.

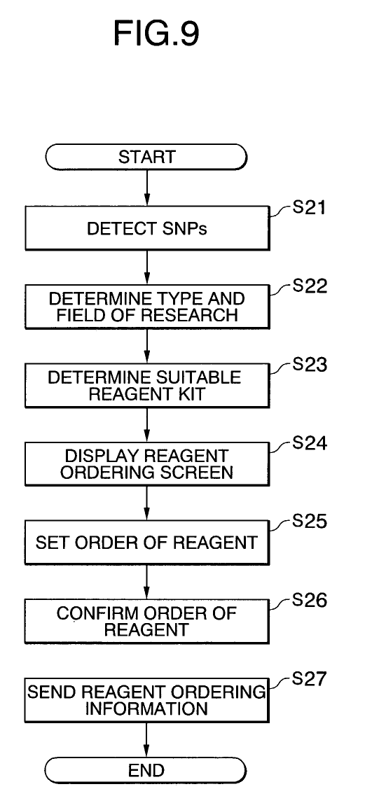

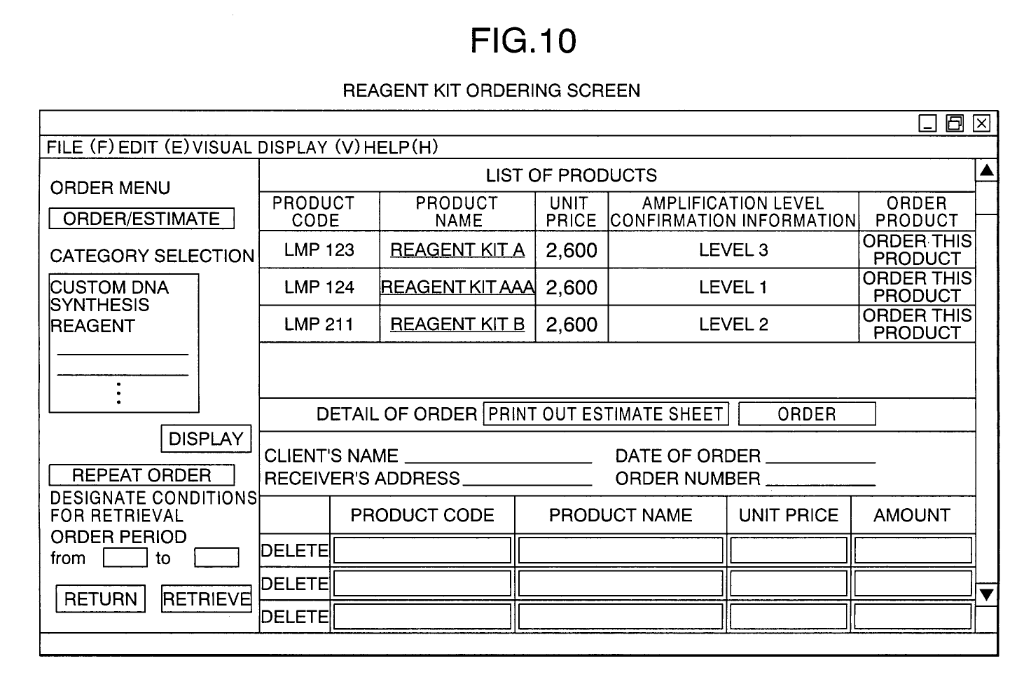

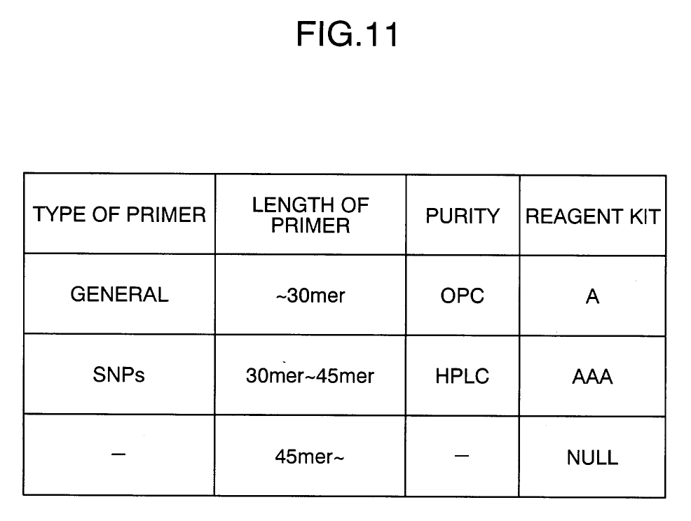

Now, based on FIG. 9, the method of ordering a reagent kit of the present invention will be explained, while referring to FIGS. 10 and 11. FIG. 9 is a view showing the flow of the reagent kit ordering processing of the present invention. After confirmation of the order of primer synthesis, where “Reagent” is selected in Category Selection on the ordering screen, the database searching means 21 of the ordering apparatus 2 searches through the DB 3 based on the base sequence of the ordered primer to detect SNPs (step S21). The term SNPs means a single-base variation in genetic information (single-nucleotide polymorphisms) and it is used as a material for determination in various researches. Then, based on the SNPs detection results, the type and field of research are considered by means of the ordering apparatus 2, and then the type and field of research is determined (step S22).

Subsequently, a reagent kit suitable with the type and field of research, which were determined from the above SNPs results, is considered on the basis of the results of the searching through the table of the DB 3 by the ordering apparatus 2. Then, a suitable reagent kit is determined (step S23), and a reagent kit ordering screen shown in FIG. 10 is displayed (step S24). An example of the reagent kit determination table of the DB3 for determining a suitable reagent kit is shown in FIG. 11. On the reagent kit determination table, correspondence between information regarding the type, length and purity of a primer, and information of a suitable reagent kit is recorded. For example, in FIG. 11, when the type of a primer is “general”, the length of the primer is 30 mer or shorter and the purity is OPC, a suitable reagent kit is determined as A. When SNPs are detected, the type of a primer is “SNPs”, the length of the primer is 30 mer to 45 mer, and the purity is HPLC, a suitable reagent kit is determined as AAA. When the length of a primer is 45 mer or longer, a Null value is shown.

On the reagent kit ordering screen in FIG. 10, the name of a reagent (product name), price (unit price) and amplification level confirmation information are default displayed in a list of products. Regarding the name of reagent, it is displayed in the order of high amplification level. The amplification level confirmation information is information regarding the image of each reagent kit, identification level and so on. For example, in respect of identification level, “Level 3” is defined as “being able to be visually confirmed under ordinary conditions”, “Level 2” is defined as “being able to be visually confirmed under certain conditions” and “Level 1” is defined as “being able to be visually confirmed under particular conditions”. The user selects a reagent kit by clicking an “Order This Product” button and then selects amount in a “Detail of Order” list, so that the order of a reagent kit is set (step S25). Then, when the user clicks an “Order” button to input an order, the order of a reagent kit is confirmed (step S26). This reagent kit ordering information is received by the input information receiving means 20 of the ordering apparatus 2 and then it is sent to the traders interested 5 via a network 4 by the ordering information sending means 23 (step S27).

According to the present invention, genome researchers can set a parameter on a portal by a selecting and clicking method according to a default display, and accordingly they can order the synthesis of a primer and a reagent kit, which are necessary and optimal for their research. As a result, it becomes possible to avoid trials and errors for setting a primer in the conventional primer designing technique, and therefore it also becomes possible to reduce the variation of research results.

What is claimed is:

1. A method of ordering synthesis of a primer used for gene amplification, the method comprising the steps of:
receiving an attribute information of a primer;
extracting a reagent information corresponding to the received attribute information from a plurality of pair of an attribute information and an reagent information corresponding to each other based on the received attribute information; and
sending the extracted reagent information.

2. A method of ordering synthesis of a primer used for gene amplification, the method comprising the steps of:
default displaying parameter information of a primer used for amplification based on input information of a target gene;
displaying the results of setting a primer used for amplification of the target gene based on selected parameter information of a primer;
default displaying primer designing information based on the selected results of setting a primer;
default displaying information regarding the type, length and purity of a primer used for amplification from the selected primer designing information; and
ordering the synthesis of a primer based on the selected information regarding the type, length and purity of a primer.

3. A method of ordering synthesis of a primer used for gene amplification according to claim 2, wherein the step of default displaying the primer designing information further comprises default displaying the information by referring order historical data in which primer synthesis ordering information is recorded.

4. A program to cause a computer to perform a processing of ordering synthesis of a primer used for gene amplification, wherein the program causes the computer to perform:
default displaying parameter information of a primer used for amplification based on input information of a target gene;
displaying the results of setting a primer used for amplification of a target gene based on the selected parameter information of a primer;
default displaying primer designing information of the basis of the selected results of setting a primer;
default displaying information regarding the type, length and purity of a primer used for amplification from the selected primer designing information; and
ordering the synthesis of a primer based on the selected information regarding the type, length and purity of a primer.

5. A computer readable recording medium recording a program to cause a computer to perform a processing of ordering synthesis of a primer used for gene amplification, wherein the program causes the computer to perform:
default displaying parameter information of a primer used for amplification based on input information of a target gene;
displaying the results of setting a primer used for amplification of a target gene based on the selected parameter information of a primer;
default displaying primer designing information of the basis of the selected results of setting a primer;
default displaying information regarding the type, length and purity of a primer used for amplification from the selected primer designing information; and
ordering the synthesis of a primer based on the selected information regarding the type, length and purity of a primer.

6. A method of ordering a reagent kit to detect gene amplification level using a primer, the method comprising the steps of:
receiving information regarding the type, length and purity of a primer ordered to be synthesized;
searching through a database table which is previously prepared based on the received information;
default displaying reagent kit information to detect gene amplification level based on the results of the searching; and
ordering a reagent kit based on reagent kit information which is selected from the default displayed contents or input separately.

7. A method of ordering a reagent kit to detect gene amplification level according to claim 6, wherein the step of default displaying reagent kit information to detect gene amplification level further comprises default displaying information of a first reagent kit and information of a second reagent kit, the information of a first reagent kit containing a primer having a high amplification level and containing amplification level confirmation information calculated based on the type, length and purity of a primer, the information of a second reagent kit containing a primer having a high amplification level in a case that a different purity is applied.

8. A method of ordering a reagent kit to detect gene amplification level according to claim 6, wherein corresponding information between information regarding the type, length and purity of a primer and reagent kit information is recorded on the database table.

9. A program to cause a computer to perform a processing of ordering a reagent kit to detect gene amplification level using a primer, wherein the program causes the computer to perform:
receiving information regarding the type, length and purity of a primer ordered to be synthesized;
searching through a database table which is previously prepared based on the received information;
default displaying reagent kit information to detect gene amplification level based on the results of the searching; and
ordering a reagent kit based on reagent kit information which is selected from the default displayed contents or input separately.

10. A computer readable recording medium recording a program to cause a computer to perform a processing of ordering a reagent kit to detect gene amplification level using a primer, wherein the program causes the computer to perform:
receiving information regarding the type, length and purity of a primer ordered to be synthesized;
searching through a database table which is previously prepared based on the received information;
default displaying reagent kit information to detect gene amplification level based on the results of the searching; and
ordering a reagent kit based on reagent kit information which is selected from the default displayed contents or input separately.

11. An apparatus for ordering synthesis of a primer and for ordering a reagent kit, the apparatus comprising:
input information receiving means for receiving parameter information necessary for the order of the synthesis of a primer and the order of a reagent kit, both of the orders being input by a user;
database searching means for searching through a database table which is previously prepared based on the received input information;
output information displaying means for default displaying output information obtained as a result of the searching process to the user's terminal; and
ordering information sending means for sending primer synthesis ordering information and reagent kit ordering information via a network, both of the information being received from the user.

12. An apparatus for ordering synthesis of a primer and for ordering a reagent kit according to claim 11, wherein corresponding information between information regarding the type, length and purity of a primer and reagent kit information is recorded on the database table.

13. A program to cause a computer to perform a primer synthesis ordering processing and a reagent kit ordering processing, wherein the program causes the computer to perform:
receiving parameter information necessary for order of synthesis of a primer and order of a reagent kit, both of the orders being input by a user;
searching through a database table which is previously prepared based on the received input information;
default displaying output information obtained as a result of the searching process to the user's terminal; and
sending primer synthesis ordering information and reagent kit ordering information via a network, both of the information being received from the user.

14. A computer readable recording medium recording a program to cause a computer to perform a primer synthesis ordering processing and a reagent kit ordering processing, wherein the program causes the computer to perform:
receiving parameter information necessary for order of synthesis of a primer and order of a reagent kit, both of the orders being input by a user;
searching through a database table which is previously prepared, based on the received input information;
displaying output information obtained as a result of the searching process to the user's terminal; and
sending primer synthesis ordering information and reagent kit ordering information via a network, both of the information being received from the user.
